(19) United States
(12) Patent Application Publication
(10) Pub. No.: US 2006/0035298 A1
(43) Pub. Date: Feb. 16, 2006

(54) System and method for determining a coagulation parameter

(76) Inventors: James Hill, Bedford (GB); Volker Unkrig, Ladenburg (DE); Ewald Rieger, Bobenheim-Roxsheim (DE)

Correspondence Address: Roche Diagnostics Corporation, 9115 Hague Road, PO Box 50457, Indianapolis, IN, 46250-0457, US

(21) Appl. No.: 11/196,534

(22) Filed: Aug. 3, 2005

Related U.S. Application Data
(63) Continuation of application No. PCT/EP04/00595, filed on Jan. 24, 2004.

(30) Foreign Application Priority Data
Feb. 1, 2003 (EP) EP 03 002 254.5

(51) Int. Cl. C12Q 1/56

(52) U.S. Cl. 435/13

(57) ABSTRACT

System and method for the determination of a coagulation parameter comprising a disposable test element and an instrument for evaluation thereof. The coagulation time is determined at a non-standard temperature different from a standard temperature for the respective coagulation parameter, the non-standard temperature being measured by a temperature measurement device of the instrument, the instrument has a non-volatile memory containing data which define a mathematical relationship of coagulation time versus temperature which relationship is independent of the individual patient whose blood is examined, and the coagulation parameter for the standard temperature is calculated from the coagulation time measured at the non-standard temperature, using the mathematical relationship.

### CROSS-REFERENCE TO RELATED APPLICATIONS

This application is a continuation of International Application No. PCT/EP2004/000595 filed 24 Jan. 2004, which claims priority to EP 03 002 254.5 filed 1 Feb. 2003, the disclosure of which is incorporated herein by reference.

### BACKGROUND OF THE INVENTION

The present invention relates to the coagulation of blood and, more particularly, to a system and method for determining a coagulation parameter.

Many medical problems relate to the coagulation of blood. In particular, during treatment with anticoagulant medication, a patient's coagulation parameters fluctuate continuously. Such fluctuations can cause severe problems. For example, if a patient is treated with an anticoagulant drug such as Heparin or Marcumar, it is important that his or her coagulation parameters remain within a defined range of values to avoid complications. Only in this way it is possible to reduce effectively the number of blood clots while simultaneously avoiding bleeding complications. A rapid, precise method for the continuous monitoring of blood coagulation parameters that meets all therapeutic needs is therefore required.

Currently, in particular three coagulation parameters are of medical interest, namely prothrombin time (PT), activated partial thromboplastin time (APTT) and activated clotting time (ACT). PT mainly serves for monitoring the effect of vitamin K antagonists on coagulation (which influence factors II, V, VII and X of the coagulation cascade). The PT test measures the activation of the extrinsic pathway by addition of tissue thromboplastin.

APTT is used primarily to monitor heparin therapy. The test detects factor changes in the intrinsic coagulation cascade (factors VIII, IX, XI, XII and other enzymes and factors). The test reagents for this type of test have not yet been standardized and therefore differences in the heparin sensitivity of reagents from different manufacturers are considerable.

ACT is determined to monitor heparinization in situations where an APTT test cannot be performed, because the patient was administered a high dose of heparin.

Traditionally, the coagulation parameters are determined by “wet chemistry” tests. An aliquot of blood sample is mixed with liquid reagents and the point of time at which the blood clots is detected. The results are indicated either directly (in seconds) or in the form of derived quantities such as ratio to a respective normal value (in percent). With respect to PT further common quantities for indication of the test results are % Quick and INR (International Normalized Ratio).

Since several years so-called “dry chemistry” tests of coagulation parameters have become available. They are performed by means of test systems comprising disposable reagent carrier elements (often designated “test elements”) and an evaluation instrument which generally is adapted for the evaluation of a particular type of test element from a particular manufacturer. The test element contains the reagent system necessary for the particular test and, typically, suitable information for the evaluation of the test such as the test type, the lot number and the expiration date.

Known coagulation test systems differ inter alia with respect to the measurable property of the coagulation detection liquid which is used to measure the coagulation time and with respect to the arrangement by which the contacting of the sample with the reagents is achieved as well as by the design of the detection zone:
In some tests the measurable property of the liquid is its viscosity which can be detected e.g., by including magnetic particles into the reagent system. The moveability of these particles can be detected by means of an alternating magnetic field. In such systems the increase of the viscosity caused by the onset of coagulation is the change marking the end of the coagulation time.
Alternatively, a chemical constituent, the concentration of which changes at a defined point of the coagulation path, can be used to mark the end of the coagulation time. In particular, the enzyme thrombin, the final protease for both plasmatic coagulation pathways, can be monitored by known means, including a reagent present in the reagent system of the test element which is suitable to generate an electrical or optical signal which can be measured by the evaluation instrument.
With respect to the physical arrangement of the test element it has been proposed to use a single permeable porous membrane which is fastened to a plastic handle similar as with traditional test strips. In such test elements the sample is directly applied to the membrane in which the reagent system is contained.
In an alternative arrangement the sample application point of the test element is located at a distance from the coagulation detection zone and the transport of the sample liquid from the former to the latter is accomplished by a capillary channel.

While tests for coagulation parameters have become relatively simple, in particular due to the introduction of dry chemistry tests, it remains an important task to further simplify the design and thus reduce the cost without sacrificing accuracy. For example, if a person received a replacement cardiac valve his or her long-term health highly depends on the fact that the coagulation status remains reliably between certain boundaries. To this end inexpensive small battery-operated instruments should be available to be used by the patients themselves in order to monitor their blood coagulation status. Similar requirements apply to “point of care (POC)” testing by the medical profession.

### SUMMARY OF THE INVENTION

It is against the above background that the present invention provides certain unobvious advantages and advancements over the prior art. In particular, the inventor has recognized a need for improvements in systems and methods for determining a coagulation parameter.

Although the present invention is not limited to specific advantages or functionality, it is noted that the present invention provides a way to simplify the design of systems for determining coagulation parameters while simultaneously maintaining the required degree of accuracy.

In accordance with one embodiment of the present invention, a system for the determination of a coagulation parameter of blood or plasma samples of patients is provided, the system comprising a disposable test element including a reagent system to be mixed with the sample, thereby forming a coagulation detection liquid, and a coagulation detection zone where the coagulation detection liquid is contained for monitoring a measurable property of the coagulation detection liquid, the measurable property changing during coagulation, and an instrument with a holder for holding the disposable test element inserted therein and with a measurement and evaluation electronics adapted for detecting a signal which corresponds to the measurable property of the coagulation detection liquid and including a time measurement electronics for measuring a coagulation time required for the change to occur for deriving the desired coagulation parameter therefrom, wherein the coagulation time is determined at a non-standard temperature of the coagulation detection liquid different from a standard temperature for the respective coagulation parameter, the non-standard temperature being measured by a temperature measurement device, the instrument has a non-volatile memory containing data which define a mathematical relationship of coagulation time versus temperature which relationship is independent of the individual patient whose blood is examined, and the coagulation parameter for the standard temperature is calculated from the coagulation time measured at the non-standard temperature, using the mathematical relationship.

In accordance with another embodiment of the present invention, a method for the determination of a coagulation parameter of a blood or plasma sample of a patient is provided comprising mixing a reagent system in a disposable test element with the sample to form a coagulation detection liquid in a reaction detection zone of the element, and monitoring a measurable property of the coagulation detection liquid, the measurable property changing during coagulation, a signal which corresponds to the measurable property of the coagulation detection liquid is detected and a time required for the change to occur is measured for deriving the desired coagulation parameter therefrom, wherein the coagulation time is determined at a non-standard temperature of the coagulation detection liquid different from a standard temperature for the respective coagulation parameter, the non-standard temperature being measured, data which define a mathematical relationship of coagulation time versus temperature are taken from a non-volatile memory of the instrument, the relationship being independent of the individual patient whose blood is examined, and the coagulation parameter for the standard temperature is calculated from the coagulation time measured at the non-standard temperature, using a mathematical relationship of coagulation time versus temperature which relationship is independent of the individual patient whose blood is examined.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a cross-sectional view of a coagulation test system with the test element inserted into the evaluation instrument in accordance with an embodiment of the present invention;

FIG. 2 shows a schematic design diagram of a system according to an embodiment of the present invention;

FIG. 3 shows an enlarged view of an element temperature sensor shown in FIG. 2;

FIG. 4 shows a graphical representation of the ratio PT³⁷/PT^{T} vs. T[° C.]/37 for a series of experiments performed in the context of the present invention;

FIG. 5 shows a graphical representation of experimental results comparing an embodiment of the present invention with a conventional determination of INR values; and

FIGS. 6 to 10 show results corresponding to FIG. 4 performed with different coagulation parameter tests.

Skilled artisans appreciate that elements in the figures are illustrated for simplicity and clarity and have not necessarily been drawn to scale. For example, the dimensions of some of the elements in the figures may be exaggerated relative to other elements to help improve understanding of the embodiment(s) of the present invention.

### DETAILED DESCRIPTION OF THE INVENTION

The present invention, in accordance with at least one embodiment, refers to dry chemistry tests. Each test element is designed to allow a drop of a blood or plasma sample (which depending on the test may be pretreated, in particular by reaction with a preparatory anticoagulant) applied thereto to contact and dissolve a reagent system (which normally comprises a plurality of reagents) present in the test element and suitable to initiate the reaction sequence of the blood coagulation path. After mixing of the sample with the reagents the resulting coagulation detection liquid is present in a coagulation detection zone of the test element. The coagulation process is monitored by observing a measurable property of the coagulation detection liquid which changes in a characteristic manner when a defined reaction step of the coagulation path occurs. The system comprises means for detecting such change and generating a corresponding signal by the measurement and evaluation electronics of the instrument. The measurement and evaluation electronics also includes a time measurement electronics for measuring a coagulation time required for the change to occur. This time is converted into the desired coagulation parameter (in the appropriate units). To this end evaluation data stored in the instrument and/or in the test element can be used. The results are then displayed on a display of the instrument and/or forwarded to further evaluation, for example by a separate computer system.

Such a system is commercially available from the applicants under the trade name CoaguChek®. Further details may be found in the appropriate literature including U.S. Pat. No. 5,789,664 and WO 01/11356, the disclosures of which are incorporated herein by reference. The sample may be whole blood or plasma. Hereafter reference is made to blood as an example. This should, however, not be understood as a limitation to the general applicability of the invention.

Known systems for the determination of coagulation parameters generally comprise some kind of thermostating device designed to maintain during the coagulation measurement a defined standard temperature, usually about 37° C. This requires an electric heater and an electronic temperature control system. Based on the present invention such a thermostating device is not necessary. Rather, the coagulation measurement can be made at a convenient temperature (e.g., about room temperature). Nevertheless, accurate values of the desired coagulation parameter can be determined which are directly comparable to those determined at the standard temperature. This leads to a plurality of advantages:
For small portable battery operated coagulation instruments the heating system is expensive and consumes more battery energy than all other components combined. The volume, weight and cost of the instruments can be reduced substantially if no heating system and a smaller battery are required.
In order to secure an exact control of the desired standard temperature, test elements of known dry chemistry systems often have a long capillary for guiding the sample liquid from the point of sample application (outside the instrument) into the interior of the instrument where the thermostating device is located. If no such long capillary is necessary the sample volume can be reduced.
The warm up time required with known instruments before testing is eliminated or reduced. This simplifies the use of the system, in particular in home monitoring or POC applications.
For INR calibration a manual technique is generally used which causes difficulties in controlling the standard (37° C.) temperature. Errors due to temperature deviations can be reduced if the technique can be performed at room temperature.

In accordance with the present invention, the change of coagulation time parameters versus temperature can be described by a functional relationship which is specific for a particular instrument and a particular reagent system but is independent on the sample. Therefore, the same functional relationship f(T) (which may e.g., be a linear function or a polynomial) describes the temperature dependence no matter whose blood is being examined. Taking PT as an example this may be expressed mathematically:
PT³⁷=f(T)*PT^{T} (1)

where

PT³⁷: PT value for the standard temperature of 37° C.

PT^{T}: PT value for a (lower) non-standard temperature.

Generally in the field of clinical chemistry it is not unusual to perform a temperature correction calculation if a test is temperature dependent and the actual test temperature differs from a desired standard test temperature. For example, tests for determining the concentration of glucose in blood are temperature dependent and it has been proposed to eliminate errors caused by temperature variations by an appropriate correction calculation.

Coagulation detection is, however, fundamentally different from the detection of the concentration of an analyte such as glucose. The temperature dependence of glucose tests is caused by influencing factors which are independent of the individual sample which is tested. In particular, the enzymatic reaction on which the test is based depends on the temperature. A coagulation test is fundamentally different since it is essentially an experimental model of the natural coagulation process which—as is well known—involves a highly complicated reaction of more than ten factors and enzymes in the blood of the particular patient. The fact that the coagulation behavior of blood is different for every individual is also apparent from the fact that the dosage of corresponding medication (e.g., Heparin or Marcumar) has to be individually adapted.

Therefore, in coagulation testing the quantity which is measured depends on the particular individual sample, i.e., the individual whose blood is tested and the status of his or her coagulation system. Regarding the temperature dependence the same had to be assumed. Therefore, it could not be expected that, by examining the blood of a limited number of patients at different temperatures with the same test system (same type of instrument and same reagent system, preferably of the same manufacturing lot) a universal f(T)-curve could be generated which allows calculation of PT³⁷ from PT^{T} values determined at a non-standard temperature for additional patients with quite different blood.

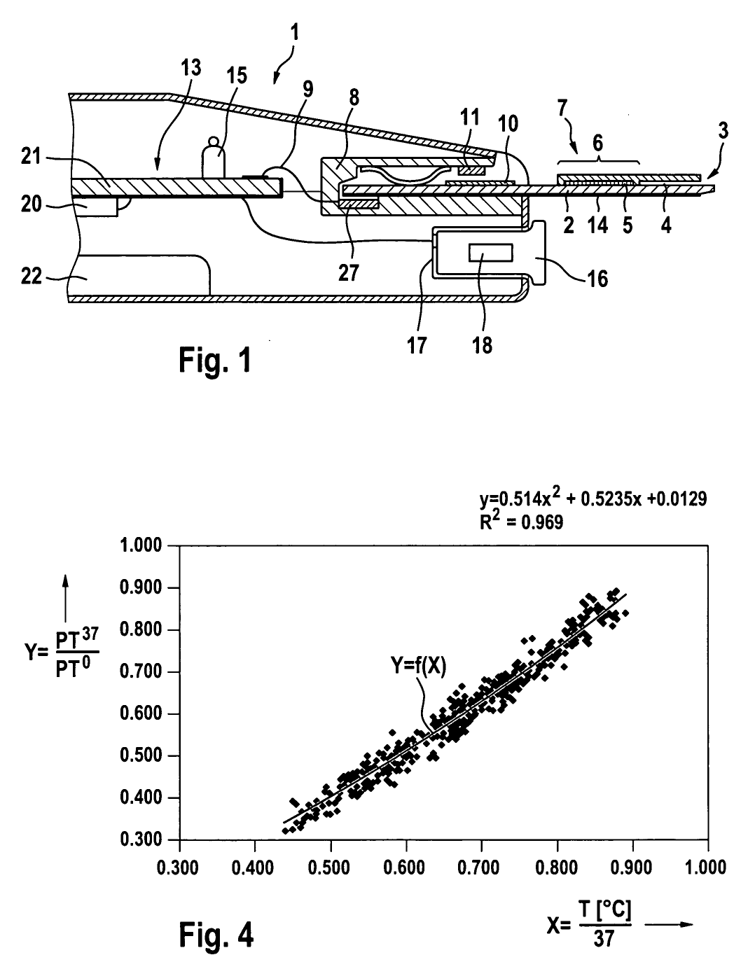

FIG. 1 is a cross-sectional schematic representation of a coagulation test system comprising an evaluation instrument 1 and a disposable test element 2, in accordance with one embodiment of the present invention. The test element has a sample application opening 3, a capillary channel 4 and a space 5 which serves as reaction chamber and coagulation detection zone 6. The channel 4 is very short and can even be omitted because with a system according to the present invention coagulation detection zone 6 need not be thermostated and is, when test element 2 is inserted in a holder 8 of instrument 1, typically located outside the housing of the instrument 1 close to sample application opening 3.

In prior art devices easy application of the sample required that the sample application opening is outside of the instrument whereas thermostating required that the coagulation detection zone of the test element is inside the instrument housing. The sample was transported from the former to the latter by a long capillary path. This again required a large sample volume not only because of the length of the path but also because of a sufficiently large capillary path cross-section required for adequate speed of liquid transport. In contrast, the present invention allows a very short transport distance (of typically less than about 1 cm or even less than about 0.5 cm) and an extremely small sample volume (typically less than about 5 μl, more typically less than about 2 μl).

Space 5 receives the coagulation detection liquid which is formed by mixing of a sample applied to the sample application opening 3 and a reagent system (not shown). Coagulation detection is performed in the coagulation detection zone 6 by detecting a change of a coagulation-related measurement quantity of the liquid contained therein. The resulting signals are transmitted via lines 9 to a measurement and evaluation unit 13 which controls the operation of the instrument. Coagulation detection can be performed by any of the methods known from the prior art, in particular by optical or electrochemical means, see e.g., U.S. Pat. No. 5,789,664 and WO 01/11356.

In accordance with a typical embodiment of the present invention, the system comprises a temperature measuring device 7 which is suitable for measuring the temperature of the coagulation detection zone 6. For example, infrared temperature detection can be used as described in WO 01/33214, the disclosure of which is incorporated herein for its teaching of infrared temperature detection. Typically, however, a temperature measurement sensor, in particular a thermistor, is integrated as element temperature sensor 14 into test element 2 and connected by plug-in contacts to the electronics of the instrument. Additionally, an instrument temperature sensor 15 may be provided and used as will be described in more detail below.

Test element 2 carries information relating to test type and reagent lot in an information field 10 which is evaluated by an element information reader 11. The detected information signal is transmitted via lines (not shown) to the measurement and evaluation unit 13. Information field 10 and information reader 11 are shown only schematically. A barcode and an appropriate barcode detector can be used as a typical example.

In accordance with the instant embodiment, a ROM key 16 sits exchangeably in a ROM key holder 17 and is connected to the central measurement and evaluation unit 13 for data exchange therewith. It includes a memory 18 in which data required for evaluation of the test are stored. These data may depend on the manufacturing lot of the test element. Typically, information field 10 contains identification data which are specific for the manufacturing lot of the particular test element 2. This information is read by information reader 11 and compared with lot identification data stored in ROM key 16 in order to make sure that the ROM key inserted into the instrument corresponds to the manufacturing lot of the test element 2.

Memory 18 of ROM key 16 can also include data describing the mathematical relationship of coagulation time versus temperature which is used for calculating the desired coagulation parameter for a standard temperature from the coagulation time measured at a non-standard temperature. Alternatively, these data may also be contained in a permanent memory of instrument 1.

The central measurement and evaluation unit 13 may consist of conventional electronic circuitry including an ASIC 20 and a circuit board 21. It comprises a programmable microprocessor for controlling the instrument functions and performing the required calculations. In particular, it combines the signal information received from coagulation detection device 6, element information device 11, non-volatile memory 18 and temperature measuring device 7 to derive the desired coagulation parameter. This result is transmitted to a display (not shown) of instrument 1. Power for the instrument operation is provided by a battery 22.

Most parts of the system shown in FIG. 1 are conventional and no further details need to be described. Deviating from the prior art, however, instrument 1 is not equipped with a thermostating system including a heater and electric heating control. Rather it uses a temperature measuring device 7 for measuring (directly or indirectly) the temperature of the coagulation detection liquid in the coagulation detection area and the mathematical relationship stored in memory 18 to calculate the coagulation parameter for the standard temperature.

In order that the invention may be more readily understood, reference is made to the following examples, which are intended to illustrate the invention, but not limit the scope thereof.

### EXAMPLES

### Example 1

Experimental evaluation of the present invention can adequately demonstrate that measuring the temperature adjacent to the space in which the coagulation detection liquid is contained is sufficient to allow accurate calculation of a desired standard coagulation parameter (which would have been obtained at a controlled temperature of approximately 37° C.) from measurements performed at a deviating (generally substantially lower) temperature using a mathematical relationship which is independent of the respective sample and can therefore be stored in a non-volatile memory of the instrument and repeatedly used for a plurality of different individuals (patients). The measurement and evaluation unit 13 is adapted to perform the required calculations.

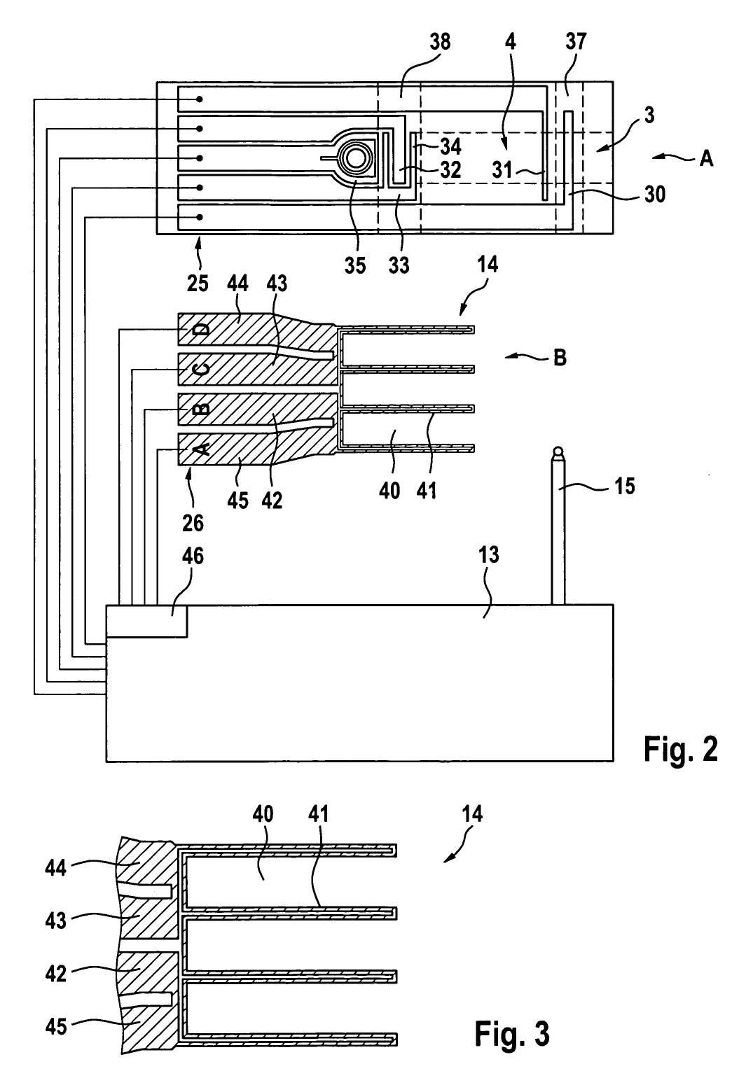

FIGS. 2 and 3 show design features of a typical embodiment of a coagulation test system partly as top view showing a typical layout of test element layers and partly as block diagram of the electronics. This embodiment is in particular characterized by two aspects:
Coagulation detection is performed by an arrangement of reagents and electrodes as shown in the upper part of FIG. 2.
The required accurate measurement of the temperature of the coagulation detection zone is achieved by the combined use of an element temperature sensor 14 integrated into the test element and an instrument temperature sensor 15 located at the instrument (relatively remote from the coagulation detection zone 6).

Both these aspects can be combined. In this case the test element typically has two layers of electric leads such as layers A and B shown in FIG. 2. Such layers can be applied in known manner onto a non-conducting (plastic) carrier foil and separated by an isolating layer. Electrical contact between the electrode arrangement provided by the leads of the layers and the instrument is achieved by element contact pads 25 and 26 respectively which provide electrical connection with corresponding instrument contacts 27 (FIG. 1). In order to provide transport of the sample liquid from a sample application opening 3 to a coagulation detection zone 6 a capillary channel 4 is provided on top of layer A. All these design elements can be embodied by known means therefore no further description thereof is required.

Electrode arrangement A of FIG. 2, in accordance with one embodiment, comprises five electrodes including a first counter electrode 30, a drop detection electrode 31, a working electrode 32, a second counter electrode 33 with two fingers 34 surrounding the working electrode 32 and a stirrup-shaped fill detection electrode 35. The electrode structure may be produced e.g., by laser ablation techniques out of gold layers of about 50 μm thickness.

Counter electrode 30 is located close to fill opening 3 and is covered by a reference reagent layer 37 containing e.g., Ag/AgCl. Working electrode 32 and second counter electrode 33 are covered by a coagulation detection reagent 38 which includes the required reagent system to start the reactions of the coagulation path. Reagent layer 38 also contains any reagents required for detection of the appropriate measurable property, in particular in the typical case of enzymatic detection of coagulation a substrate of the respective enzyme, for example the substrate electrozyme TH of the enzyme thrombin.

A test protocol performed with the system shown in FIG. 2 may include the following steps:
The instrument is switched on automatically by inserting a test element and usual operation checks are performed.
When a sample drop is applied to sample opening 3 of capillary channel 4 and the liquid bridges first counter electrode 30 and drop detection electrode 31 this may be detected by applying a suitable AC-potential to these electrodes and detecting a sharp drop of the impedance when both electrodes are contacted by the liquid.
When the blood sample is further drawn into capillary channel 4 (by capillary action) its arrival at working electrode 32 and second counter electrode 33 can again be detected by applying a suitable AC-potential to these electrodes. The wetting of working electrode 32 is also a suitable starting point for the coagulation time measurement as it coincides with dissolving of reagent layer 38 and therefore with the start of the coagulation reaction sequence.
Complete fill of capillary 4 is detected by a current flowing between fill detection electrode 34 and second counter electrode 33.
An enzyme activity is detected as measurable property of the coagulation detection liquid by electrochemical means. To this end after complete fill of capillary 4 a suitable constant DC-potential is applied to working electrode 32 with reference to first counter electrode 30. Thereafter current measurements are taken at suitable intervals (e.g., about 0.1 seconds). When at the end of the coagulation path the enzyme Thrombin is formed it cleaves an electrochemically active group from the substrate contained in reagent layer 38 thereby causing an increase of the current which is characteristic of the enzyme formation. This change defines the end of the coagulation time which is used for deriving the desired coagulation parameter in known manner.

Layer B shown in FIG. 2 is an example of electric leads suitable as element temperature sensor 14. Its temperature sensitive range 40 is defined by the area in which a very thin and narrow electric thermistor-conductor runs, typically in a meandering manner as shown in more detail in FIG. 3. Conductor 41 can be made from typical thermistor material with a large dependence of resistance versus temperature. However, also materials more common for test element manufacturing including a gold layer of sufficiently small thickness (less than about 100 μm) can be suitable.

The resistance of the thermistor-conductor 41 can be measured by a four-terminal arrangement where two terminals 42,43 are used for feeding a constant current into thermistor-conductor 41 and two separate terminals 44,45 are used to measure the resistance in current-free manner.

During operation of the system the temperature signal generated by element temperature sensor 14 can be used in combination with the signal generated by instrument sensor 15 to determine a reliable temperature value of high accuracy to be used for calculation of the coagulation parameter for the standard temperature from a measurement performed at a (lower) non-standard temperature:
The element temperature sensor 14 is only used to provide information about temperature changes in close proximity of the coagulation detection zone 6. It is therefore not necessary for this sensor and the temperature measurement electronics 46 (which is part of measurement and evaluation electronics 13) to allow temperature measurement in absolute quantities.
Instrument temperature sensor 15 can be used to provide temperature information relative to the standard temperature. This temperature information has to be absolute in the sense that the measured difference from the standard temperature is (with a high degree of accuracy) the same for all instruments which is a necessary requirement for using the same mathematical relationship of coagulation time versus temperature for the temperature conversion of all instruments.

In practical use, after insertion of a test element 2 into instrument 1 the temperature of element temperature sensor 14 can be monitored to derive an information about the change of the element temperature versus time or in other words the speed of element temperature change. Only when this speed of change falls below an acceptable value (in other words only when the temperature of the element 2 is sufficiently constant) a signal is given that a drop of a sample may be applied to the test element to perform a determination of coagulation parameter. If this condition is met the temperature value of instrument temperature sensor 15 is used as “true” temperature for the conversion.

Typically, not only the temperature change of element temperature sensor 14 but also the temperature change of instrument temperature sensor 15 is monitored. Even better results are achieved if a coagulation determination takes place only when the speed of change of both temperature sensors is below a suitable limit (which may be different for both sensors) indicating a highly constant temperature environment.

Experimental evaluation of the present invention shows that by these means a temperature measurement is possible which fulfills even the extremely high requirements of coagulation tests. Simultaneously due to the fact that the test element temperature sensor need not provide absolute temperature values it can be incorporated relatively inexpensively into the disposable test elements. The instrument temperature sensor 15 needs not be located inside the instrument. Rather, it can be even advantageous to locate sensor 15 at the outside of the instrument in a position where the environmental temperature is measured.

FIG. 4 shows experimental results which can be generated as follows:
Samples from 27 different patients and four standard liquids (“normals”) are taken, each separated in multiple aliquots.
From each sample four measurements of PT^{T}-values are made at four non-standard temperatures between about 16° and about 32° C. (using four aliquots). These measurements can be performed with a CoaguCheck®S instrument as manufactured by the applicant which is modified by disabling its thermostating system. Temperature variation can be provided by positioning the instrument in a temperature chamber.
In order to improve the precision and increase the data base each of these measurements can be performed with four different instruments.
Simultaneously for each sample PT³⁷ can be determined using a conventional CoaguCheck®S system with thermostating.

From the results PT-ratios Y=PT³⁷/PT^{T} can be calculated. These PT-ratios are shown in FIG. 2 relative to a temperature ratio X calculated by dividing the respective measurement temperature T (in ° C.) by 37.

FIG. 4 shows that all data are very close to a curve Y=f(X) determined by regression analysis therefrom. In this case the curve is a second order polynomial as shown in FIG. 4. This proves the surprising fact that the temperature dependence can be described by a single functional relationship independent on the source of the samples used.

With respect to the temperature it can be convenient during practical experiments to use temperature ratios (as shown in place of the absolute temperatures). Evidently Y=f(X) can be easily transformed to Y=PT³⁷/PT^{T}=f(T). Furthermore, an equation for calculating PT³⁷ is easily found by solving this equation for PT³⁷:
PT³⁷=f(T)*PT^{T}

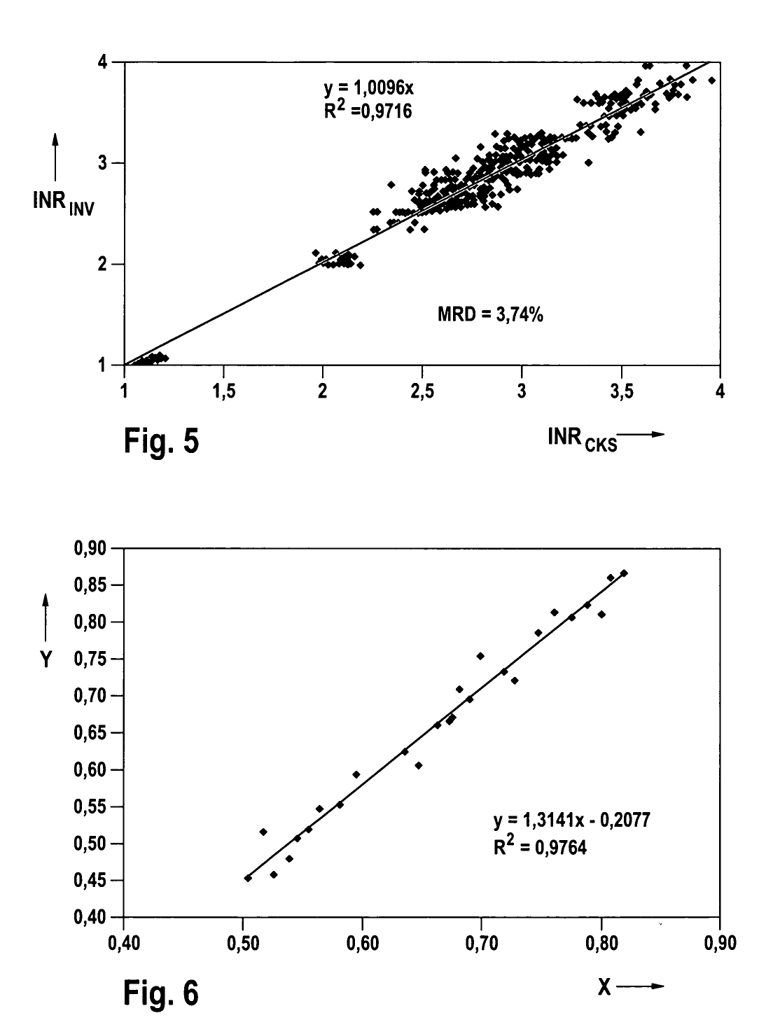

FIG. 5 shows a comparison of INR-values determined according to the invention (designated INR_{INV}) with corresponding data determined by a reference method (INR_{CKS}). INR values are calculated by forming a ratio between PT and a Median Normal PT (MNPT) as is well known in the art. The figure shows that the results generated by both methods are in agreement with a Mean Relative Deviation (MRD) of 3.74%. It should be noted that the values of INR_{INV} can be determined with ambient temperature measurements between about 16° C. and about 32° C. whereas the reference values INR_{CKS} can be determined at the standard temperature of about 37° C.

FIGS. 6 to 10 show the results of experiments that can be performed with a smaller number of samples but a plurality of different methods. In each case a ratio of a coagulation parameter at a standard temperature and the same parameter at the respective non-standard temperature (designated Y for PT-ratios, Z for INR-ratios and V for APTT-ratios) is plotted against the temperature ratio X. The individual figures are based on experiments using the following coagulation test systems:

FIG. 6: CoaguCheck®S Low ISI PT Strips. These tests use dry Low ISI thromboplastin derived from human recombinant tissue factor requiring non-anticoagulated fresh venous of capillary blood. The samples were from patients on oral anticoagulant treatment.

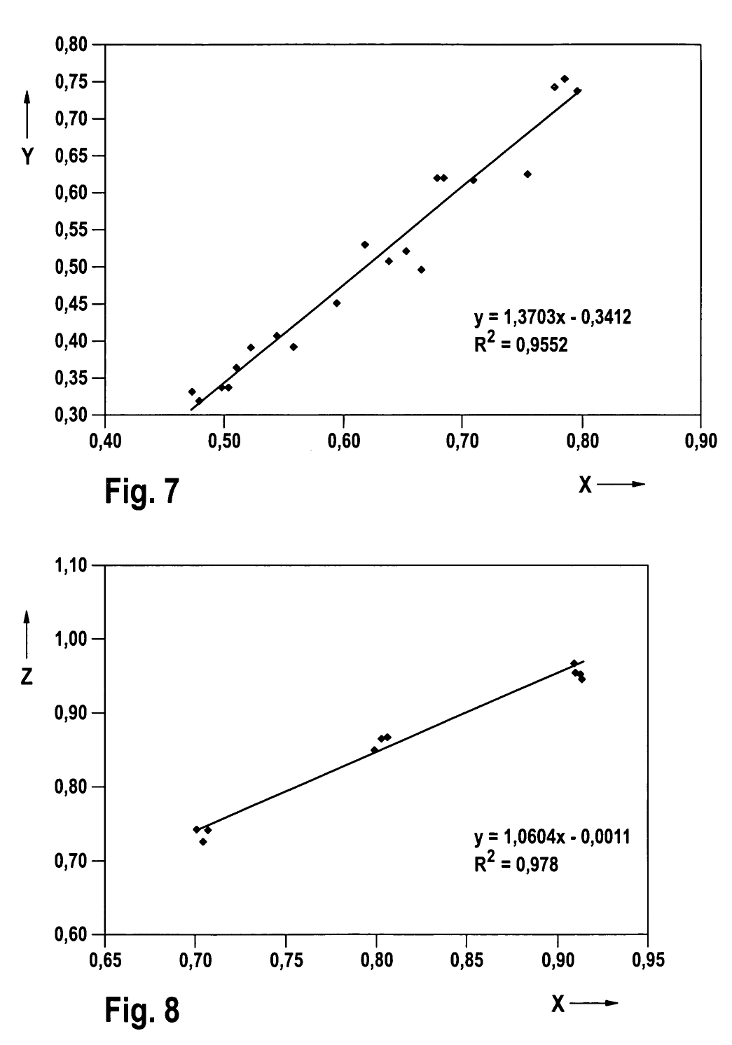

FIG. 7: CoaguCheck® S Low Volume PT Strips. These strips contain High ISI thromboplastin derived from rabbit brains requiring non-anticoagulated fresh venous or capillary blood.

FIG. 8: Amelung 4 Channel Lab Analyzer with Low ISI Lab reagent. This reagent contains Ortho Recombiplastin PT derived from human recombinant tissue factor requiring citrated plasma.

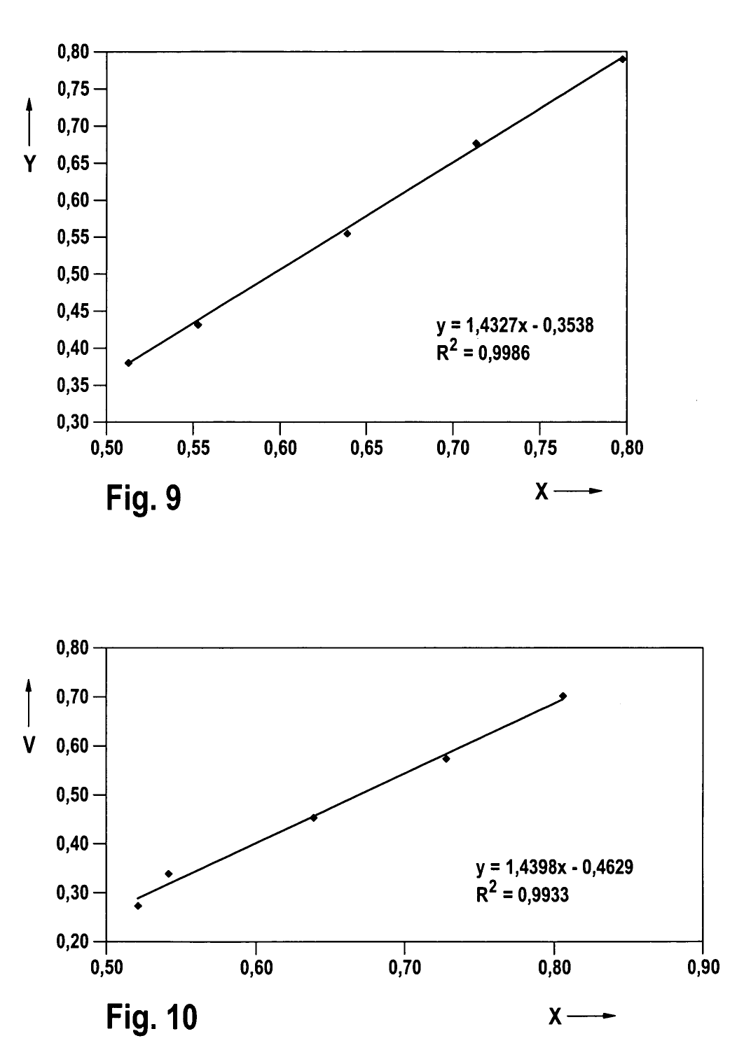

FIG. 9: Amelung 4 Channel Lab Analyzer with High ISI Lab reagent. This reagent includes Dade C Plus PT derived from rabbit brains requiring citrated plasma.

FIG. 10: Amelung 4 Channel Lab Analyzer with Ortho Auto APTT Lab reagent requiring citrated plasma.

In each case the resulting data can be described for all samples by a single functional relationship as shown in the figures. Thus the invention is applicable to different types of coagulation tests including both “dry chemistry” and “wet chemistry” test.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A system for the determination of a coagulation parameter of blood or plasma samples of patients, said system comprising:
a disposable test element including a reagent system to be mixed with the sample, thereby forming a coagulation detection liquid, and a coagulation detection zone where the coagulation detection liquid is contained for monitoring a measurable property of the coagulation detection liquid, said measurable property changing during coagulation, and
an instrument with a holder for holding said disposable test element inserted therein and with a measurement and evaluation electronics adapted for detecting a signal which corresponds to the measurable property of the coagulation detection liquid and including a time measurement electronics for measuring a coagulation time required for said change to occur for deriving the desired coagulation parameter therefrom, wherein
the coagulation time is determined at a non-standard temperature of the coagulation detection liquid different from a standard temperature for the respective coagulation parameter, said non-standard temperature being measured by a temperature measurement device,
the instrument has a non-volatile memory containing data which define a mathematical relationship of coagulation time versus temperature which relationship is independent of the individual patient whose blood is examined, and
the coagulation parameter for the standard temperature is calculated from the coagulation time measured at said non-standard temperature, using said mathematical relationship.

2. The system according to claim 1, wherein the instrument contains no device for thermostating the test element with the coagulation detection liquid contained therein, whereby the temperature of the test element and of the coagulation detection liquid depends on the temperature of the environment in which the measurement is performed.

3. The system according to claim 1, wherein the instrument is battery-operated.

4. The system according to claim 1, wherein
the disposable test element includes an element temperature sensor integrated therein,
the instrument includes an instrument temperature sensor the measurement and evaluation electronics is adapted to derive
from the signal of the instrument temperature sensor a temperature value relative to the standard temperature and
from the signal of the element temperature sensor an information about the speed of change of the element temperature versus time and
the measurement and evaluation electronics uses the temperature value derived from the instrument temperature sensor for calculating the coagulation parameter for said standard temperature from the coagulation time measured at said non-standard temperature only when the speed of change of the element temperature versus time is smaller than a limiting value thereof.

5. The system according to claim 1, wherein the coagulation detection zone is outside the instrument housing when the test element is inserted in the holder.

6. The system according to claim 1, wherein the instrument comprises an interchangeable machine readable data storage element, said interchangeable data storage element including the memory containing the data which define the mathematical relationship of coagulation time versus temperature.

7. The system according to claim 6, wherein the interchangeable machine readable data storage element is a ROM key.

8. The system according to claim 6 wherein the test element comprises a machine readable code containing identification data which are specific for the manufacturing lot of the test element and the instrument has a reader for reading said machine readable identification code for checking by the measurement and evaluation electronics that a ROM key inserted into the instrument corresponds to the manufacturing lot of the test element.

9. A test element adapted for the system according to claim 1.

10. The test element according to claim 9, wherein said test element is disposable.

11. The test element according to claim 9 comprising a temperature measurement sensor and plug-in contacts allowing transfer of temperature signals of the temperature sensor to an instrument into which it is inserted.

12. The test element according to claim 11, wherein said temperature measurement sensor is a thermistor.

13. The system according to claim 1, wherein the measurable property is the activity of an enzyme which participates in the coagulation pathway of the coagulation detection liquid.

14. The system according to claim 13, wherein the activity of the enzyme is detected by electrochemical means.

15. The system according to claim 1, wherein the same mathematical relationship is used for all determinations of a given coagulation parameter performed with the instrument, independent of the reagent lot.

16. The system according to claim 1, wherein the mathematical relationship is determined by measuring the coagulation time for a plurality of different samples at different temperatures using the same type of instrument and reagents.

17. A method for the determination of a coagulation parameter of a blood or plasma sample of a patient, comprising:
mixing a reagent system in a disposable test element with the sample to form a coagulation detection liquid in a reaction detection zone of the element, and
monitoring a measurable property of the coagulation detection liquid, said measurable property changing during coagulation,
a signal which corresponds to the measurable property of the coagulation detection liquid is detected and a time required for said change to occur is measured for deriving the desired coagulation parameter therefrom, wherein the coagulation time is determined at a non-standard temperature of the coagulation detection liquid different from a standard temperature for the respective coagulation parameter, said non-standard temperature being measured,
data which define a mathematical relationship of coagulation time versus temperature are taken from a non-volatile memory of the instrument, said relationship being independent of the individual patient whose blood is examined, and
the coagulation parameter for the standard temperature is calculated from the coagulation time measured at said non-standard temperature, using a mathematical relationship of coagulation time versus temperature which relationship is independent of the individual patient whose blood is examined.

18. The method according to claim 17, wherein the measurable property is the activity of an enzyme which participates in the coagulation pathway of the coagulation detection liquid.

19. The method according to claim 18, wherein the activity of the enzyme is detected by electrochemical means.

20. The method according to claim 17, wherein the same mathematical relationship is used for all determinations of a given coagulation parameter performed with the instrument, independent of the reagent lot.

21. The method according to claim 17, wherein the mathematical relationship is determined by measuring the coagulation time for a plurality of different samples at different temperatures using the same type of instrument and reagents.
